(19) United States
(12) Patent Application Publication
(10) Pub. No.: US 2026/0014351 A1
(43) Pub. Date: Jan. 15, 2026

(54) OPEN AND CLOSE TORQUE LIMITING ACTUATOR FOR EMD TORQUER

(71) Applicant: Corindus, Inc., Newton, MA (US)

(72) Inventors: Christopher Zirps, Sharon, MA (US); Zilong Huang, Billerica, MA (US); Peter Falb, Hingham, MA (US)

(21) Appl. No.: 18/769,591

(22) Filed: Jul. 11, 2024

(51) Int. Cl. A61M 25/01; A61B 17/00; A61B 90/00

(52) U.S. Cl. CPC A61M 25/0113; A61B 2017/00292; A61B 2090/031; A61M 2025/0166

(57) ABSTRACT

A torquer releasably securing an elongated medical device thereto and including a torque limiting actuator limiting a torque applied to the torquer in a fully open position.

### CROSS-REFERENCE TO RELATED PATENT APPLICATIONS

None.

### FIELD

The present invention relates generally to the field of robotic medical procedure systems and, in particular, to a torquer for elongated medical devices.

### BACKGROUND

Catheters and other elongated medical devices (EMDs) may be used for minimally invasive medical procedures for the diagnosis and treatment of diseases of various vascular systems, including neurovascular intervention (NVI) also known as neurointerventional surgery, percutaneous coronary intervention (PCI) and peripheral vascular intervention (PVI). These procedures typically involve navigating a guidewire through the vasculature, and via the guidewire advancing a catheter to deliver therapy. The catheterization procedure starts by gaining access into the appropriate vessel, such as an artery or vein, with an introducer sheath using standard percutaneous techniques. Through the introducer sheath, a sheath or guide catheter is then advanced over a diagnostic guidewire to a primary location such as an internal carotid artery for NVI, a coronary ostium for PCI, or a superficial femoral artery for PVI. A guidewire suitable for the vasculature is then navigated through the sheath or guide catheter to a target location in the vasculature. In certain situations, such as in tortuous anatomy, a support catheter or microcatheter is inserted over the guidewire to assist in navigating the guidewire. The physician or operator may use an imaging system (e.g., fluoroscope) to obtain a cine with a contrast injection and select a fixed frame for use as a roadmap to navigate the guidewire or catheter to the target location, for example, a lesion. Contrast-enhanced images are also obtained while the physician delivers the guidewire or catheter so that the physician can verify that the device is moving along the correct path to the target location. While observing the anatomy using fluoroscopy, the physician manipulates the proximal end of the guidewire or catheter to direct the distal tip into the appropriate vessels toward the lesion or target anatomical location and avoid advancing into side branches.

Robotic catheter-based procedure systems have been developed that may be used to aid a physician in performing catheterization procedures such as, for example, NVI, PCI and PVI. Examples of NVI procedures include coil embolization of aneurysms, liquid embolization of arteriovenous malformations and mechanical thrombectomy of large vessel occlusions in the setting of acute ischemic stroke. In an NVI procedure, the physician uses a robotic system to gain target lesion access by controlling the manipulation of a neurovascular guidewire and microcatheter to deliver the therapy to restore normal blood flow. Target access is enabled by the sheath or guide catheter but may also require an intermediate catheter for more distal territory or to provide adequate support for the microcatheter and guidewire. The distal tip of a guidewire is navigated into, or past, the lesion depending on the type of lesion and treatment. For treating aneurysms, the microcatheter is advanced into the lesion and the guidewire is removed and several embolization coils are deployed into the aneurysm through the microcatheter and used to block blood flow into the aneurysm. For treating arteriovenous malformations, a liquid embolic is injected into the malformation via a microcatheter. Mechanical thrombectomy to treat vessel occlusions can be achieved either through aspiration and/or use of a stent retriever. Depending on the location of the clot, aspiration is either done through an aspiration catheter, or through a microcatheter for smaller arteries. Once the aspiration catheter is at the lesion, negative pressure is applied to remove the clot through the catheter. Alternatively, the clot can be removed by deploying a stent retriever through the microcatheter. Once the clot has integrated into the stent retriever, the clot is retrieved by retracting the stent retriever and microcatheter (or intermediate catheter) into the guide catheter.

In PCI, the physician uses a robotic system to gain lesion access by manipulating a coronary guidewire to deliver the therapy and restore normal blood flow. The access is enabled by seating a guide catheter in a coronary ostium. The distal tip of the guidewire is navigated past the lesion and, for complex anatomies, a microcatheter may be used to provide adequate support for the guidewire. The blood flow is restored by delivering and deploying a stent or balloon at the lesion. The lesion may need preparation prior to stenting, by either delivering a balloon for pre-dilation of the lesion, or by performing atherectomy using, for example, a laser or rotational atherectomy catheter and a balloon over the guidewire. Diagnostic imaging and physiological measurements may be performed to determine appropriate therapy by using imaging catheters or fractional flow reserve (FFR) measurements.

In PVI, the physician uses a robotic system to deliver the therapy and restore blood flow with techniques similar to NVI. The distal tip of the guidewire is navigated past the lesion and a microcatheter may be used to provide adequate support for the guidewire for complex anatomies. The blood flow is restored by delivering and deploying a stent or balloon to the lesion. As with PCI, lesion preparation and diagnostic imaging may be used as well.

When support at the distal end of a catheter or guidewire is needed, for example, to navigate tortuous or calcified vasculature, to reach distal anatomical locations, or to cross hard lesions, an over-the-wire (OTW) catheter or coaxial system is used. An OTW catheter has a lumen for the guidewire that extends the full length of the catheter. This provides a relatively stable system because the guidewire is supported along the whole length. This system, however, has some disadvantages, including higher friction, and longer overall length compared to rapid-exchange catheters (see below). Typically to remove or exchange an OTW catheter while maintaining the position of the indwelling guidewire, the exposed length (outside of the patient) of guidewire must be longer than the OTW catheter. A 300 cm long guidewire is typically sufficient for this purpose and is often referred to as an exchange length guidewire. Due to the length of the guidewire, two operators are needed to remove or exchange an OTW catheter. This becomes even more challenging if a triple coaxial, known in the art as a tri-axial system, is used (quadruple coaxial catheters have also been known to be used). However, due to its stability, an OTW system is often used in NVI and PVI procedures. On the other hand, PCI procedures often use rapid exchange (or monorail) catheters. The guidewire lumen in a rapid exchange catheter runs only through a distal section of the catheter, called the monorail or rapid exchange (RX) section. With a RX system, the operator manipulates the interventional devices parallel to each other (as opposed to with an OTW system, in which the devices are manipulated in a serial configuration), and the exposed length of guidewire only needs to be slightly longer than the RX section of the catheter. A rapid exchange length guidewire is typically 180-200 cm long. Given the shorter length guidewire and monorail, RX catheters can be exchanged by a single operator. However, RX catheters are often inadequate when more distal support is needed.

### SUMMARY

In some aspects, the techniques described herein relate to a device for manipulating an elongated medical device including: a torquer releasably securing an elongated medical device (EMD) thereto; and a torque limiting actuator limiting a torque applied to the torquer in a fully open position.

In some aspects, the techniques described herein relate to a torquer for an elongated medical device: a body having a cavity defining a pathway; a first pad movable within the cavity; a biasing member separate from the first pad biasing the first pad relative to the body; an actuator movable relative to the body moving the first pad, wherein movement of a first pad pinches and/or unpinches the elongated medical device, with the first pad, within the pathway; and a knob releasably connected to a shaft of the actuator upon an application of a predetermined torque exceeding a predetermined value in a fully open position, wherein the shaft is movable to a fixed position relative to an actuator body in the fully open position.

In some aspects, the techniques described herein relate to an EMD drive system including: a robotic drive having a robotic drive longitudinal axis; a device module movable along the robotic drive longitudinal axis; and a drive train coupling a motor to a driven member configured to rotate a torquer pinching an elongated medical device (EMD) about an EMD longitudinal axis, the torquer including a torque limiting actuator being manually accessible to a user when the torquer is in an in-use position in the drive module; wherein the torque limiting actuator limits a torque applied to the torquer in a fully open position.

### BRIEF DESCRIPTION OF THE DRAWINGS

FIG. 1 is a schematic view of an exemplary catheter procedure system.

FIG. 2 is a schematic block diagram of an exemplary catheter procedure system.

FIG. 3 is an exploded view of a cassette assembly and robotic drive and drive modules of a catheter procedure system.

FIG. 4 is an isometric view of a torquer actuator.

FIG. 5 is an exploded view of the torquer actuator of FIG. 4.

FIG. 6 is a side plan view of the torquer actuator of FIG. 4.

FIG. 7 is a cross sectional view of the torquer actuator of FIG. 6 in the open position.

FIG. 7A is a cross sectional view of the torquer actuator of FIG. 6 in the closed position.

FIG. 8 is an exploded view of a torque limiting knob assembly of the torquer actuator of FIG. 4.

FIG. 8A is a close-up for a portion of the torque limiting assembly.

FIG. 8B is an isometric view of the knob of the torque limiting assembly.

FIG. 9 is an isometric view of the pads and biasing member of the torquer actuator of FIG. 4.

FIG. 10 is an isometric view of the torquer actuator in a cassette of the catheter procedure system.

FIG. 11 is a cross-sectional view of a torquer actuator illustrating various stop features.

### DETAILED DESCRIPTION OF THE EXAMPLE EMBODIMENTS

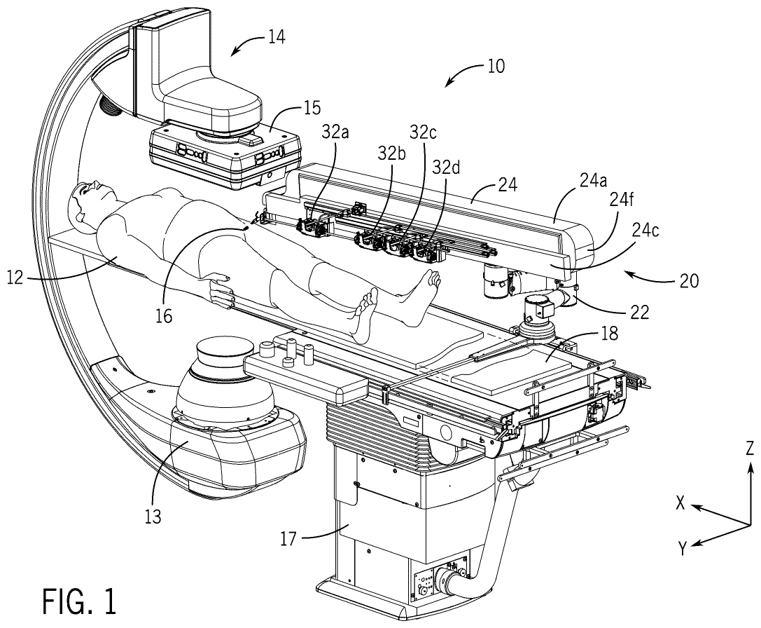

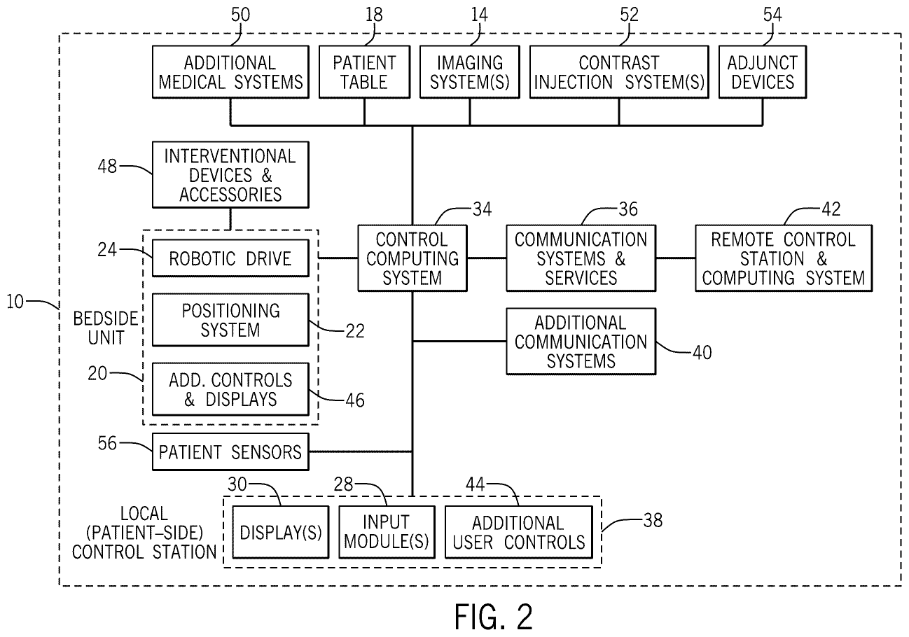

FIG. 1 is a perspective view of an example catheter-based procedure system 10 in accordance with an embodiment. Catheter-based procedure system 10 may be used to perform catheter-based medical procedures, e.g., percutaneous intervention procedures such as a percutaneous coronary intervention (PCI) (e.g., to treat STEMI), a neurovascular interventional procedure (NVI) (e.g., to treat an emergent large vessel occlusion (ELVO)), peripheral vascular intervention procedures (PVI) (e.g., for critical limb ischemia (CLI), etc.). Catheter-based medical procedures may include diagnostic catheterization procedures during which one or more catheters or other elongated medical devices (EMDs) are used to aid in the diagnosis of a patient's disease. For example, during one embodiment of a catheter-based diagnostic procedure, a contrast in media is injected onto one or more arteries through a catheter and an image of the patient's vasculature is taken. Catheter-based medical procedures may also include catheter-based therapeutic procedures (e.g., angioplasty, stent placement, treatment of peripheral vascular disease, clot removal, arterial venous malformation therapy, treatment of aneurysm, etc.) during which a catheter (or other EMD) is used to treat a disease. Therapeutic procedures may be enhanced by the inclusion of adjunct devices 54 (shown in FIG. 2) such as, for example, intravascular ultrasound (IVUS), optical coherence tomography (OCT), fractional flow reserve (FFR), etc. It should be noted, however, that one skilled in the art would recognize that certain specific percutaneous intervention devices or components (e.g., type of guidewire, type of catheter, etc.) may be selected based on the type of procedure that is to be performed. Catheter-based procedure system 10 can perform any number of catheter-based medical procedures with minor adjustments to accommodate the specific percutaneous intervention devices to be used in the procedure.

Catheter-based procedure system 10 includes, among other elements, a bedside unit 20 and a control station (not shown). Bedside unit 20 includes a robotic drive 24 and a positioning system 22 that are located adjacent to a patient 12. Patient 12 is supported on a patient table 18. The positioning system 22 is used to position and support the robotic drive 24. The positioning system 22 may be, for example, a robotic arm, an articulated arm, a holder, etc. The positioning system 22 may be attached at one end to, for example, the patient table 18 (as shown in FIG. 1), a base, or a cart. The other end of the positioning system 22 is attached to the robotic drive 24. The positioning system 22 may be moved out of the way (along with the robotic drive 24) to allow for the patient 12 to be placed on the patient table 18. Once the patient 12 is positioned on the patient table 18, the positioning system 22 may be used to situate or position the robotic drive 24 relative to the patient 12 for the procedure. The position of the robotic drive in a position for the procedure is referred to herein as the robotic drive in-use position. In an embodiment, patient table 18 is operably supported by a pedestal 17, which is secured to the floor and/or earth. Patient table 18 is able to move with multiple degrees of freedom, for example, roll, pitch, and yaw, relative to the pedestal 17. Bedside unit 20 may also include controls and displays 46 (shown in FIG. 2). For example, controls and displays may be located on a housing of the robotic drive 24.

Generally, the robotic drive 24 may be equipped with the appropriate percutaneous interventional devices and accessories 48 (shown in FIG. 2) (e.g., guidewires, various types of catheters including but not limited to balloon catheters, stent delivery systems, stent retrievers, embolization coils, liquid embolics, aspiration pumps, device to deliver contrast media, medicine, hemostasis valve adapters, syringes, stopcocks, inflation device, etc.) to allow a user or operator to perform a catheter-based medical procedure via a robotic system by operating various controls such as the controls and inputs located at the control station. Bedside unit 20, and in particular robotic drive 24, may include any number and/or combination of components to provide bedside unit 20 with the functionality described herein. The robotic drive 24 includes a plurality of device modules 32a-d mounted to a rail or linear member. Each of the device modules 32a-d may be used to drive an EMD such as a catheter or guidewire. For example, the robotic drive 24 may be used to automatically feed a guidewire into a diagnostic catheter and into a guide catheter in an artery of the patient 12. One or more devices, such as an EMD, enter the body (e.g., a vessel) of the patient 12 at an insertion point 16 via, for example, an introducer sheath. Each device module 32a-d include a drive module and cassette removably attached to the drive module. Each drive module is movable along the robotic drive longitudinal axis with a bracket or stage. While FIG. 1 illustrates four device modules it is contemplated that the number of device modules may be one or more.

Bedside unit 20 is in communication with the control station (not shown), allowing signals generated by the user inputs of the control station to be transmitted wirelessly or via hardwire to the bedside unit 20 to control various functions of bedside unit 20. As discussed below, control station may include a control computing system 34 (shown in FIG. 2) or be coupled to the bedside unit 20 through the control computing system 34. Bedside unit 20 may also provide feedback signals (e.g., loads, speeds, operating conditions, warning signals, error codes, etc.) to the control station, control computing system 34 (shown in FIG. 2), or both. Communication between the control computing system 34 and various components of the catheter-based procedure system 10 may be provided via a communication link that may be a wireless connection, cable connections, or any other means capable of allowing communication to occur between components. The control station or other similar control system may be located either at a local site (e.g., local control station 38 shown in FIG. 2) or at a remote site (e.g., remote control station and computer system 42 shown in FIG. 2). Catheter procedure system 10 may be operated by a control station at the local site, a control station at a remote site, or both the local control station and the remote control station at the same time. At a local site, a user or operator and the control station are located in the same room or an adjacent room to the patient 12 and bedside unit 20. As used herein, a local site is the location of the bedside unit 20 and a patient 12 or subject (e.g., animal or cadaver) and the remote site is the location of a user or operator and a control station used to control the bedside unit 20 remotely. A control station (and a control computing system) at a remote site and the bedside unit 20 and/or a control computing system at a local site may be in communication using communication systems and services 36 (shown in FIG. 2), for example, through the Internet. In an embodiment, the remote site and the local (patient) site are away from one another, for example, in different rooms in the same building, different buildings in the same city, different cities, or other different locations where the remote site does not have physical access to the bedside unit 20 and/or patient 12 at the local site.

The control station generally includes one or more input modules 28 configured to receive user inputs to operate various components or systems of catheter-based procedure system 10. In the embodiment shown, control station allows the user or operator to control bedside unit 20 to perform a catheter-based medical procedure. For example, input modules 28 may be configured to cause bedside unit 20 to perform various tasks using percutaneous intervention devices (e.g., EMDs) interfaced with the robotic drive 24 (e.g., to advance, retract, or rotate a guidewire, advance, retract or rotate a catheter, inflate or deflate a balloon located on a catheter, position and/or deploy a stent, position and/or deploy a stent retriever, position and/or deploy a coil, inject contrast media into a catheter, inject liquid embolics into a catheter, inject medicine or saline into a catheter, aspirate on a catheter, or to perform any other function that may be performed as part of a catheter-based medical procedure). Robotic drive 24 includes various drive mechanisms to cause movement (e.g., axial and rotational movement) of the components of the bedside unit 20 including the percutaneous intervention devices.

In one embodiment, input modules 28 may include one or more touch screens, joysticks, scroll wheels, and/or buttons. In addition to input modules 28, the control station may use additional user controls 44 (shown in FIG. 2) such as foot switches and microphones for voice commands, etc. Input modules 28 may be configured to advance, retract, or rotate various components and percutaneous intervention devices such as, for example, a guidewire, and one or more catheters or microcatheters. Buttons may include, for example, an emergency stop button, a multiplier button, device selection buttons and automated move buttons. When an emergency stop button is pushed, the power (e.g., electrical power) is shut off or removed to bedside unit 20. When in a speed control mode, a multiplier button acts to increase or decrease the speed at which the associated component is moved in response to a manipulation of input modules 28. When in a position control mode, a multiplier button changes the mapping between input distance and the output commanded distance. Device selection buttons allow the user or operator to select which of the percutaneous intervention devices loaded into the robotic drive 24 are controlled by input modules 28. Automated move buttons are used to enable algorithmic movements that the catheter-based procedure system 10 may perform on a percutaneous intervention device without direct command from the user or operator. In one embodiment, input modules 28 may include one or more controls or icons (not shown) displayed on a touch screen (that may or may not be part of a display), that, when activated, causes operation of a component of the catheter-based procedure system 10. Input modules 28 may also include a balloon or stent control that is configured to inflate or deflate a balloon and/or deploy a stent. Each of the input modules 28 may include one or more buttons, scroll wheels, joysticks, touch screen, etc. that may be used to control the particular component or components to which the control is dedicated. In addition, one or more touch screens may display one or more icons (not shown) related to various portions of input modules 28 or to various components of catheter-based procedure system 10.

Catheter-based procedure system 10 also includes an imaging system 14. Imaging system 14 may be any medical imaging system that may be used in conjunction with a catheter based medical procedure (e.g., non-digital X-ray, digital X-ray, CT, MRI, ultrasound, etc.). In an exemplary embodiment, imaging system 14 is a digital X-ray imaging device that is in communication with the control station. In one embodiment, imaging system 14 may include a C-arm (shown in FIG. 1) that allows imaging system 14 to partially or completely rotate around patient 12 in order to obtain images at different angular positions relative to patient 12 (e.g., sagittal views, caudal views, anterior-posterior views, etc.). In one embodiment imaging system 14 is a fluoroscopy system including a C-arm having an X-ray source 13 and a detector 15, also known as an image intensifier.

Imaging system 14 may be configured to take X-ray images of the appropriate area of patient 12 during a procedure. For example, imaging system 14 may be configured to take one or more X-ray images of the head to diagnose a neurovascular condition. Imaging system 14 may also be configured to take one or more X-ray images (e.g., real time images) during a catheter-based medical procedure to assist the user or operator of the control station to properly position a guidewire, guide catheter, microcatheter, stent retriever, coil, stent, balloon, etc. during the procedure. The image or images may be displayed on display 30. For example, images may be displayed on a display to allow the user or operator to accurately move a guide catheter or guidewire into the proper position.

In order to clarify directions, a rectangular coordinate system is introduced with X, Y, and Z axes. The positive X axis is oriented in a longitudinal (axial) distal direction, that is, in the direction from the proximal end to the distal end, stated another way from the proximal to distal direction. The Y and Z axes are in a transverse plane to the X axis, with the positive Z axis oriented up, that is, in the direction opposite of gravity, and the Y axis is automatically determined by right-hand rule. As used herein the X axis extends along a longitudinal axis of the robotic drive 24. Since in an in-use position the robotic housing may be at an angle with respect to the horizontal plane perpendicular to the direction of gravity the X, Y and Z axes are defined by robotic drive 24. Referring to FIG. 1, robotic drive 24 includes a housing having a top or first member 24a parallel to the X-Y plane; a bottom or second member parallel to and spaced from the first member 24a; a front or third member 24c substantially perpendicular and extending between first member 24a and the second member, the third member facing a user when robotic drive 24 is in the in-use position or orientation illustrated in FIG. 1. A fourth member is spaced from and substantially parallel to third member 24c and perpendicular to first member 24a and the second member. It is contemplated that other shapes of the robotic drive housing may be used. In which case first member 24a would be the upper member, the second member would be the lower or bottom member, front or third member 24c would be the portion facing a user in an in-use position during a surgical procedure, and the fourth member is the portion facing away from the user in the in-use position during a surgical procedure. Robotic drive 24 further includes a distal region 24e and a proximal region 24f. Where the distal region 24c is closer to the entry point of the patient through which the EMD will be introduced and the proximal region 24f is furthest from the entry point of the patient through which the EMD will be introduced.

FIG. 2 is a block diagram of catheter-based procedure system 10 in accordance with an example embodiment. Catheter-procedure system 10 may include a control computing system 34. Control computing system 34 may physically be, for example, part of a control station. Control computing system 34 may generally be an electronic control unit suitable to provide catheter-based procedure system 10 with the various functionalities described herein. For example, control computing system 34 may be an embedded system, a dedicated circuit, a general-purpose system programmed with the functionality described herein, etc. Control computing system 34 is in communication with bedside unit 20, communications systems and services 36 (e.g., Internet, firewalls, cloud services, session managers, a hospital network, etc.), a local control station 38, additional communications systems 40 (e.g., a telepresence system), a remote control station and computing system 42, and patient sensors 56 (e.g., electrocardiogram (ECG) devices, electroencephalogram (EEG) devices, blood pressure monitors, temperature monitors, heart rate monitors, respiratory monitors, etc.). The control computing system is also in communication with imaging system 14, patient table 18, additional medical systems 50, contrast injection systems 52 and adjunct devices 54 (e.g., IVUS, OCT, FFR, etc.). The bedside unit 20 includes a robotic drive 24, a positioning system 22 and may include additional controls and displays 46. As mentioned above, the additional controls and displays may be located on a housing of the robotic drive 24. Interventional devices and accessories 48 (e.g., guidewires, catheters, etc.) interface to the bedside unit 20. In an embodiment, interventional devices and accessories 48 may include specialized devices (e.g., IVUS catheter, OCT catheter, FFR wire, diagnostic catheter for contrast, etc.) which interface to their respective adjunct devices 54, namely, an IVUS system, an OCT system, and FFR system, etc.

In various embodiments, control computing system 34 is configured to generate control signals based on the user's interaction with input modules 28 (e.g., of a control station such as a local control station 38 or a remote control station 42) and/or based on information accessible to control computing system 34 such that a medical procedure may be performed using catheter-based procedure system 10. The local control station 38 includes one or more displays 30, one or more input modules 28, and additional user controls 44. The remote control station and computing system 42 may include similar components to the local control station 38. The remote 42 and local 38 control stations can be different and tailored based on their required functionalities. The additional user controls 44 may include, for example, one or more foot input controls. The foot input control may be configured to allow the user to select functions of the imaging system 14 such as turning on and off the X-ray and scrolling through different stored images. In another embodiment, a foot input device may be configured to allow the user to select which devices are mapped to scroll wheels included in input modules 28. Additional communication systems 40 (e.g., audio conference, video conference, telepresence, etc.) may be employed to help the operator interact with the patient, medical staff (e.g., angio-suite staff), and/or equipment in the vicinity of the bedside.

Catheter-based procedure system 10 may be connected or configured to include any other systems and/or devices not explicitly shown. For example, catheter-based procedure system 10 may include image processing engines, data storage and archive systems, automatic balloon and/or stent inflation systems, medicine injection systems, medicine tracking and/or logging systems, user logs, encryption systems, systems to restrict access or use of catheter-based procedure system 10, etc.

As mentioned, control computing system 34 is in communication with bedside unit 20 which includes a robotic drive 24, a positioning system 22 and may include additional controls and displays 46 and may provide control signals to the bedside unit 20 to control the operation of the motors and drive mechanisms used to drive the percutaneous intervention devices (e.g., guidewire, catheter, etc.). The various drive mechanisms may be provided as part of a robotic drive 24.

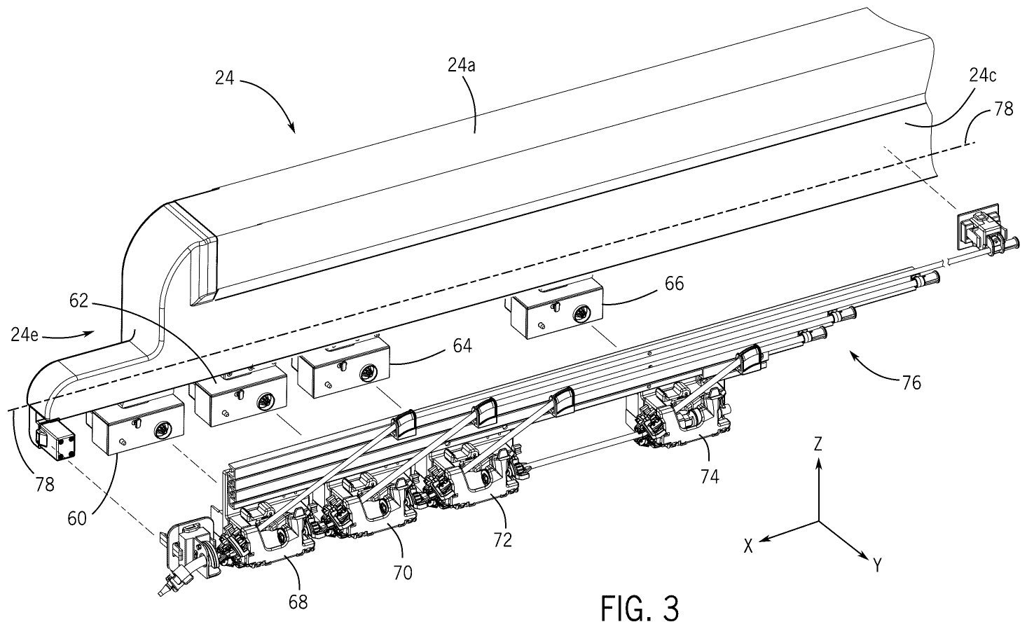

Referring to FIG. 3 device module 32a includes a first drive module 60 and a first cassette 68. Device module 32b includes a second drive module 62 and a second cassette 70. Device module 32c includes a third drive module 64 and a third cassette 72. Device module 32d includes a fourth drive module 66 and a fourth cassette 74. In one implementation first cassette 68, second cassette 70, third cassette 72 and fourth cassette 74 are shipped together as a multi-unit cassette assembly. In one implementation the multi-unit cassette assembly 76 allows for each of the cassettes to be removably connected to their respective drive modules while slidably connected together. In one implementation each of the multiple device modules 32a-d may be independently actuated to move linearly along a linear member within robotic drive 24. Each device module 32a-d may independently move relative to each other and the linear member in the robotic drive. The drive mechanism moves each device module along a longitudinal axis 78 of robotic drive 24, also referred to herein as the robotic drive longitudinal axis 78. Robotic drive longitudinal axis 78 may extend along the linear member such as a screw drive along which device modules move or may be defined along another axis that is parallel to the linear member along which the device modules move. Referring to FIG. 3 each cassette 68-74 are generally vertically oriented in the XZ plane. Each cassette 68-74 has a length along the X axis or parallel to longitudinal axis 78 that is greater than the width of each cassette along the Y axis or perpendicular to the Y axis. PCT International Publication No. WO 2021/011533, which is incorporated herein by reference in its entirety, discloses a cassette positioned in a generally horizontal position in the XY plane. The distinction between the vertical and horizontal of the cassettes is described in PCT International Publication No. WO 2021/011554 and incorporated herein by reference in its entirety.

Referring to WO 2021/011554, in one implementation, the drive mechanism includes independent stage translation motors coupled to each device module and a stage drive mechanism such as a lead screw via a rotating nut, a rack via a pinion, a belt via a pinion or pulley, a chain via a sprocket, or the stage translation motors 64a-d may be linear motors themselves. The drive mechanism provides for advancement and retraction of the device modules. Examples of such drive mechanisms are described in WO 2021/011533.

To prevent contaminating the patient with pathogens, healthcare staff use aseptic techniques in a room housing the bedside unit 20 and the patient 12 or subject (shown in FIG. 1). A room housing the bedside unit 20 and patient 12 may be, for example, a cath lab or an angio suite. Aseptic technique consists of using sterile barriers, sterile equipment, proper patient preparation, environmental controls and contact guidelines. Accordingly, all EMDs and interventional accessories are sterilized and can only be in contact with either sterile barriers or sterile equipment. In an embodiment, a sterile drape (not shown) is placed over the non-sterile robotic drive 24. Each cassette 68-74 is sterilized and acts as a sterile interface between the draped robotic drive 24 and at least one EMD. Each cassette 68-74 can be designed to be sterile for single use or to be re-sterilized in whole or part so that the cassette 68-74 or its components can be used in multiple procedures.

Distal and Proximal: The terms distal and proximal define relative locations of two different features. With respect to a robotic drive the terms distal and proximal are defined by the position of the robotic drive in its intended use relative to a patient. When used to define a relative position, the distal feature is the feature of the robotic drive that is closer to the patient than a proximal feature when the robotic drive is in its intended in-use position. Within a patient, any vasculature landmark further away along the path from the access point is considered more distal than a landmark closer to the access point, where the access point is the point at which the EMD enters the patient. Similarly, the proximal feature is the feature that is farther from the patient than the distal feature when the robotic drive in its intended in-use position. When used to define direction, the distal direction refers to a path on which something is moving or is aimed to move or along which something is pointing or facing from a proximal feature toward a distal feature and/or patient when the robotic drive is in its intended in-use position. The proximal direction is the opposite direction of the distal direction. By way of examples referring to FIG. 1, a robotic device is shown from the viewpoint of an operator facing a patient. In this arrangement, the distal direction is along the positive X coordinate axis and the proximal direction is along the negative X coordinate axis.

Longitudinal axis: The term longitudinal axis of a member (for example, an EMD or other element in the catheter-based procedure system) is the line or axis along the length of the member that passes through the center of the transverse cross section of the member in the direction from a proximal portion of the member to a distal portion of the member. For example, the longitudinal axis of a guidewire is the central axis in the direction from a proximal portion of the guidewire toward a distal portion of the guidewire even though the guidewire may be non-linear in the relevant portion.

Axial Movement: The term axial movement of a member refers to translation of the member along the longitudinal axis of the member. When the distal end of an EMD is axially moved in a distal direction along its longitudinal axis into or further into the patient, the EMD is being advanced. When the distal end of an EMD is axially moved in a proximal direction along its longitudinal axis out of or further out of the patient, the EMD is being withdrawn.

Rotational Movement: The term rotational movement of a member refers to the change in angular orientation of the member about the local longitudinal axis of the member. Rotational movement of an EMD corresponds to clockwise or counterclockwise rotation of the EMD about its longitudinal axis due to an applied torque.

Axial and Lateral Insertion: The term axial insertion refers to inserting a first member into a second member along the longitudinal axis of the second member. An EMD that is axially loaded in a collet is axially inserted in the collet. An example of axial insertion could be referred to as back loading a catheter on the proximal end of a guidewire. The term lateral insertion refers to inserting a first member into a second member along a direction in a plane perpendicular to the longitudinal axis of the second member. This can also be referred to as radial loading or side loading. Stated another way, lateral insertion refers to inserting a first member into a second member along a direction that is parallel to the radius and perpendicular to the longitudinal axis of the second member.

Up/Down; Front/Rear; Inwardly/Outwardly: The terms top, up, and upper refer to the general direction away from the direction of gravity and the terms bottom, down, and lower refer to the general direction in the direction of gravity. The term front refers to the side of the robotic drive that faces a bedside user and away from the positioning system, such as the articulating arm. The term rear refers to the side of the robotic drive that is closest to the positioning system, such as the articulating arm. The term inwardly refers to the inner portion of a feature. The term outwardly refers to the outer portion of a feature.

Stage: The term stage refers to a member, feature, or device that is used to couple a device module to the robotic drive. For example, the stage may be used to couple the device module to a rail or linear member of the robotic drive.

Drive Module: The term drive module generally refers to the part (e.g., the capital part) of the robotic drive system that normally contains one or more motors with drive couplers that interface with the cassette.

Device Module: The term device module refers to the combination of a drive module and a cassette.

Cassette: The term cassette generally refers to the part (non-capital, consumable or sterilizable unit) of the robotic drive system that normally is the sterile interface between a drive module and at least one EMD (directly) or through a device adapter (indirectly).

Shaft (Distal) Driving: The term shaft (distal) driving refers to holding on to and manipulating an EMD along its shaft. In one example the on-device adapter is normally placed just proximal of the hub or Y-connector the device is inserted into. If the location of the on-device adapter is at the proximity of an insertion point (to the body or another catheter or valve), shaft driving does not typically require anti-buckling features. (It may include anti-buckling features to improve drive capability.)

Collet: The term collet refers to a device that can releasably fix a portion of an EMD. The term fixed here means no intentional relative movement of the collet and EMD during operation. In one embodiment the collet includes at least two members that move rotationally relative to each other to releasably fix the EMD to at least one of the two members. In one embodiment the collet includes at least two members that move axially (along a longitudinal axis) relative to each other to releasably fix the EMD to at least one of the two members. In one embodiment the collet includes at least two members that move rotationally and axially relative to each other to releasably fix the EMD to at least one of the two members.

Fixed: The term fixed means no intentional relative movement of a first member with respect to a second member during operation.

Pinch/Unpinch: The term pinch refers to releasably fixing an EMD to a member such that the EMD and member move together when the member moves. The term unpinch refers to releasing the EMD from a member such that the EMD is no longer fixed to a member but unfixed to that member and the EMD moves independently of the member.

On-Device Adapter: The term on-device adapter refers to a sterile apparatus capable of releasably pinching an EMD to provide a driving interface. The on-device adapter is also known as an end-effector or EMD capturing device. In one non-limiting embodiment the on-device adapter is a collet that is operatively controlled robotically to rotate the EMD about its longitudinal axis, to pinch and/or unpinch the EMD to the collet, and/or to translate the EMD along its longitudinal axis. In one embodiment the on-device adapter is a hub-drive mechanism such as a gear located on the hub of an EMD.

EMD: The term elongated medical device (EMD) refers to, but is not limited to, catheters (e.g., guide catheters, microcatheters, balloon/stent catheters), wire-based devices (e.g., guidewires, embolization coils, stent retrievers, etc.), and medical devices comprising any combination of these. In one example a wire-based EMD includes but is not limited to guidewires, microwires, a proximal pusher for embolization coils, stent retrievers, self-expanding stents, and flow divertors. Typically wire-based EMD's do not have a hub or handle at its proximal terminal end. In one embodiment the EMD is a catheter having a hub at a proximal end of the catheter and a flexible shaft extending from the hub toward the distal end of the catheter, wherein the shaft is more flexible than the hub. In one embodiment the catheter includes an intermediary portion that transitions between the hub and the shaft that has an intermediate flexibility that is less rigid than the hub and more rigid than the shaft. In one embodiment the intermediary portion is a strain relief.

Hub (Proximal) Driving: The term hub driving, or proximal driving refers to holding on to and manipulating an EMD from a proximal position (e.g., a geared adapter on a catheter hub). In one embodiment, hub driving refers to imparting a force or torque to the hub of a catheter to translate and/or rotate the catheter. Hub driving may cause the EMD to buckle and thus hub driving often requires anti-buckling features. For devices that do not have hubs or other interfaces (e.g., a guidewire), device adapters may be added to the device to act as an interface for the device module. In one embodiment, an EMD does not include any mechanism to manipulate features within the catheter such as wires that extend from the handle to the distal end of the catheter to deflect the distal end of the catheter.

Sterilizable Unit: The term sterilizable unit refers to an apparatus that is capable of being sterilized (free from pathogenic microorganisms). This includes, but is not limited to, a cassette, consumable unit, drape, device adapter, and sterilizable drive modules/units (which may include electromechanical components). Sterilizable Units may come into contact with the patient, other sterile devices, or anything else placed within the sterile field of a medical procedure.

Sterile Interface: The term sterile interface refers to an interface or boundary between a sterile and non-sterile unit. For example, a cassette may be a sterile interface between the robotic drive and at least one EMD.

Consumable: The term consumable refers to a sterilizable unit that normally has a single use in a medical procedure. The unit could be a reusable consumable through a re-sterilization process for use in another medical procedure.

Gear: The term gear may be a bevel gear, spiral bevel gear, spur gear, miter gear, worm gear, helical gear, rack and pinon, screw gear, internal gear such as a sun gear, involute spline shafts and bushing, or any other type of gears known in the art.

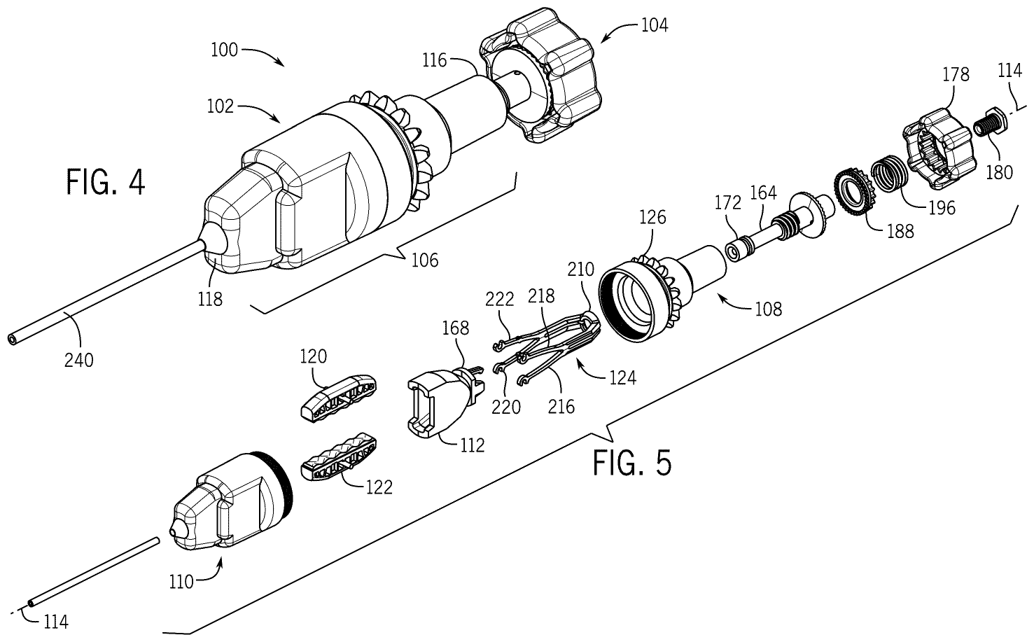

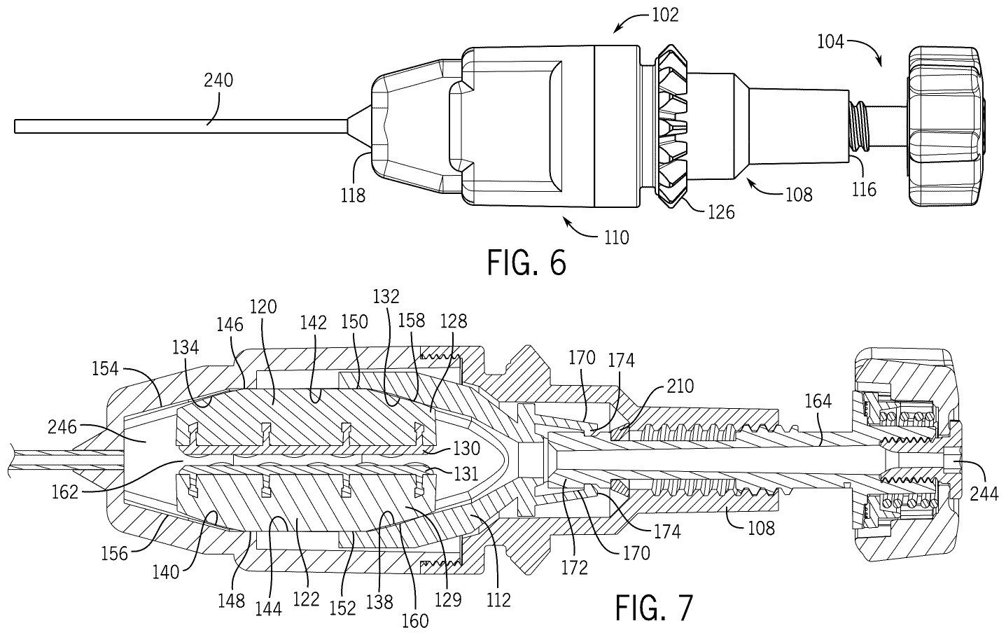

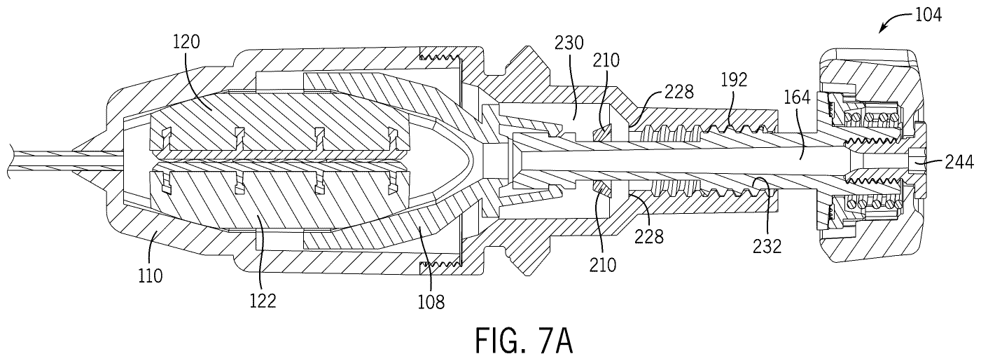

Referring to FIG. 4 and FIG. 5 a torquer actuator 100 includes a torquer 102 and a torque limiting actuator 104. Torquer actuator 100 includes a housing 106 formed from a proximal housing member 108 that is operatively connected to a distal housing member 110. Housing 106 is also referred to herein as body 106. A pusher 112 is movably received within housing 106 along a torquer longitudinal axis 114 between a proximal end 116 of housing and distal end 118 of housing 106. A first pad 120 and a second pad 122 move toward and away from torquer longitudinal axis 114 to releasably pinch a shaft of an elongated medical device (EMD). A pad biasing member 124 biases first pad 120 and second pad 122 away from one another. Movement of pusher 112 from the proximal end 116 toward distal end 118 forces first pad 120 and second pad 122 toward one another and provides sufficient force to overcome the biasing force of pad biasing member 124. Movement of pusher 112 from distal end 118 toward proximal end 116 permits pad biasing member 124 to bias first pad 120 and second pad 122 away from one another and away from torquer longitudinal axis 114. Referring to FIG. 7 in one implementation first pad 120 includes a first portion 128 and a second portion 130 contacting the EMD in an engaged position.

First portion 128 of first pad 120 includes an outer surface having a proximal ramp 132 and a distal ramp 134. Similarly, second pad 122 includes a first portion 129 and a second portion 131 that contacts the EMD in an engaged position. First portion 129 of second pad 122 includes a proximal ramp 138 and a distal ramp 140.

First pad 120 includes a first land portion 142 (also referred to as a first leveling surface) intermediate proximal ramp 132 and distal ramp 134. Second pad 122 includes a second land portion 144 (also referred to as a second leveling surface) intermediate proximal ramp 138 and distal ramp 140. First land portion 142 and second land portion 144 are parallel to one another and parallel to torquer longitudinal axis 114.

Distal housing member 110 includes a first land surface 146 and a second land surface 148. First land surface 146 and second land surface 148 are parallel to one another and parallel to torquer longitudinal axis 114 and parallel to first land portion 142 of first pad 120 and second land portion 144 of second pad 122.

Pusher 112 includes a first land surface 150 and a second land surface 152. First land surface 150 and second land surface 152 are parallel to one another and parallel to torquer longitudinal axis 114. In one implementation first land surface 146 of distal housing member 110 and first land surface 150 of pusher 112 are coplanar. Similarly, second land surface 148 of distal housing member 110 and second land surface 152 of pusher 112 are coplanar.

Distal housing member 110 includes a first ramp 154 and a second ramp 156. Both first ramp 154 and second ramp 156 have a non-parallel orientation to torquer longitudinal axis 114. Pusher 112 includes a first ramp 158 and a second ramp 160. Referring to FIG. 7A, as pusher 112 is moved from a proximal position toward a distal position, first ramp 158 and second ramp 160 of pusher 112 contacts proximal ramp 132 and proximal ramp 138 of first pad 120 and second pad 122 respectively. Similarly, distal ramp 134 and distal ramp 140 contacts first ramp 154 and second ramp 156 of distal housing member 110 respectively.

Referring to FIG. 7, as Pusher 112 is moved from the distal position toward the proximal position pad biasing member 124 biases first pad 120 and second pad 122 away from one another and away from torquer longitudinal axis 114. First pad 120 maintains a general parallel orientation torquer longitudinal axis 114 by contact of first land portion 142 of first pad 120 to first land surface 146 of distal housing member 110 and to first land surface 150 of pusher 112. Similarly, second pad 122 maintains a general parallel orientation torquer longitudinal axis 114 by contact of second land portion 144 to second land surface 148 of distal housing member 110 and second land surface 152 of pusher 112. In this way first pad 120 and second pad 122 remain parallel to one another allowing for a pathway 162 between second portion 130 of first pad 120 and second portion 131 of second pad 122 permitting easy insertion and removal of an EMD within pathway 162.

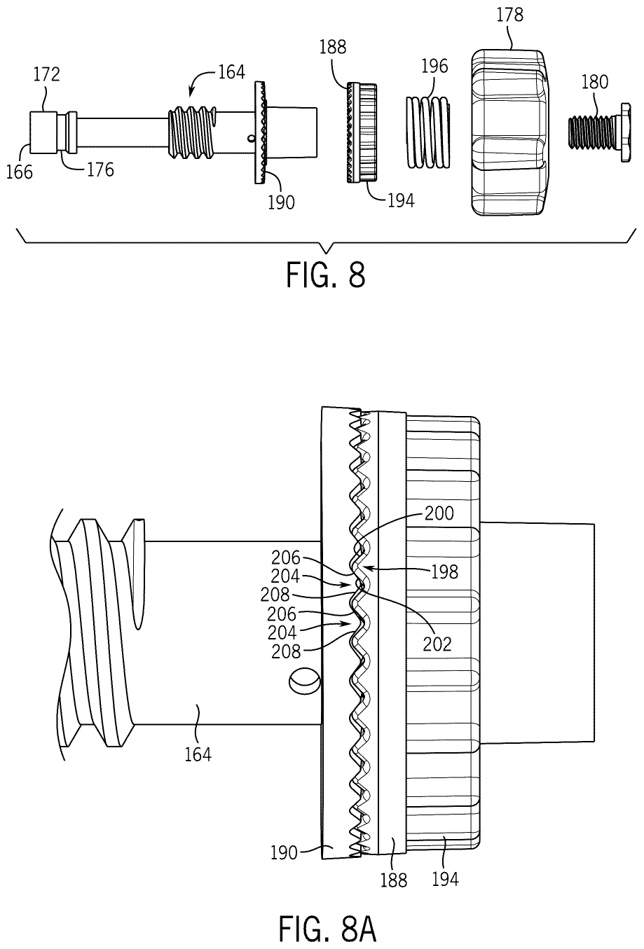

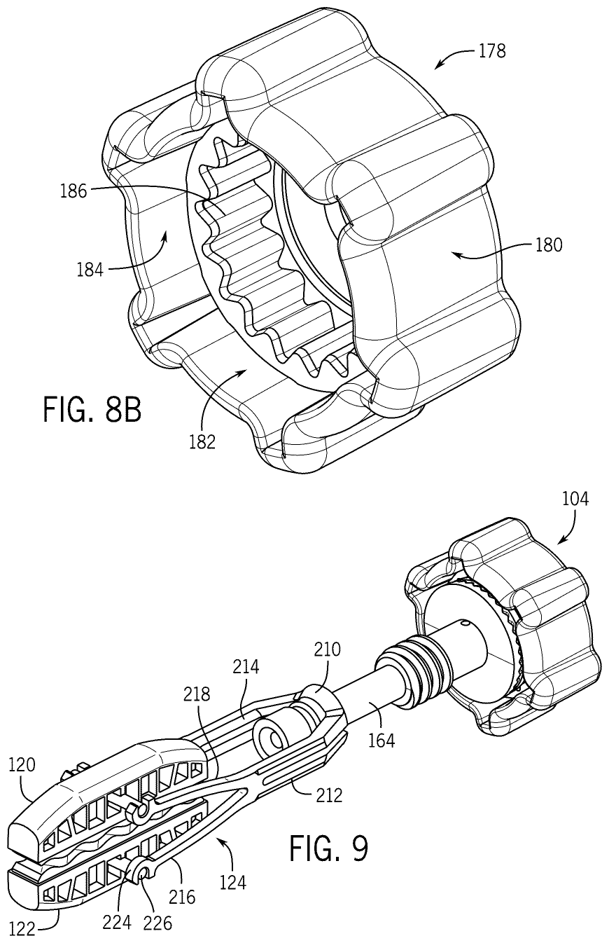

Pusher 112 is moved distally within distal housing member 110 by manipulation of torque limiting actuator 104. Referring to FIG. 7 and FIG. 8 torque limiting actuator 104 includes a shaft 164 that is threadedly engaged with proximal housing member 108. Shaft 164 includes a distal end 166 that is operatively connected to pusher 112 such that movement of shaft 164 in a distal direction moves pusher 112 in a distal direction and movement of shaft 164 in a proximal direction moves pusher 112 in a proximal direction. Pusher 112 includes a connector portion 168 having a pair of arms 170 that engage a distal hub 172 of shaft 164 such that when shaft 164 is moved in a proximal direction, pusher 112 also moves in a proximal direction. Each arm 170 includes a tab 174 that is received within a groove 176 of distal hub 172 of shaft 164. Arms 170 are resilient such that tab 174 snap fits within groove 176. The term snap fit is well known in the art and refers to an assembly method used to attach flexible parts, usually plastic, to form the final product by pushing the parts' interlocking components together.

Torque limiting actuator 104 includes a knob 178 that is secured to shaft 150 with a fastener 180. Referring to FIG. 8B, knob 178 includes an outer surface 180 that an operator manipulates and an interior cavity 182 that is defined by a cavity wall 184. In one implementation cavity wall 184 includes a profile defined by a plurality of spaced ribs 186 that positively engage with a profile on a drive gear 188 that is positioned within cavity 182 and rotates with knob 178. Shaft 150 includes a driven gear 190 that is engaged with drive gear 188. Rotation of knob 178 in a first direction results in rotation of drive gear 188 which in turn rotates driven gear 190 and shaft 150 in a first direction. As knob 178 is rotated in the first direction shaft 150 moves in a distal direction within the threaded region 192 of proximal housing member 108. Movement in the distal direction then moves pusher 112 in a distal direction which causes first pad 120 and second pad 122 to move toward one another to pinch the EMD. In one implementation the profile of the cavity wall is a plurality of splines 186 that engage with mating splines 194 of a housing supporting drive gear 172. The mating splines prohibit rotational movement between knob 178 and drive gear 188 but allow for axial movement along torquer longitudinal axis 114 between knob 178 and drive gear 188.

A biasing member 196 is positioned within cavity 182 of knob 178 and acts to bias drive gear 188 into engagement with driven gear 190. Referring to FIG. 8A driven gear 190 and drive gear 188 are face gears in that they face one another and rotate about a common axis, in this case, drive gear 188 and driven gear 190 rotate about torquer longitudinal axis 114. Drive gear 188 includes a plurality of gear teeth 198 having a first face 200 and a second face 202 where the angle of first face 200 is the same as the angle of second face 202. In one implementation the angle of first face 200 is equal to or greater than the angle of second face 202. Similarly, driven gear 190 has a plurality of gear teeth 204 having a first face 206 and a second face 208 that engage with first face 200 and second face 202 of gear teeth 198.

Drive gear 188 and driven gear 190 act as a clutch, wherein drive gear 188 is a first clutch plate and driven gear 190 is a second clutch plate. When the torque exceeds a predetermined value first face 200 of gear teeth 198 rides up and over a corresponding first face 206 of gear teeth 204 resulting in drive gear 188 slipping with respect to driven gear 190. Stated another way the manner in which the gear teeth ride over one another rather than stay engaged during the application of a predetermined torque is referred to herein as slip or slipping. In one implementation the angle of first face 206 and the angle of second face 208 is complimentary to the angle of first face 200 and second face 202 respectively. In one implementation, drive gear 188 and driven gear 190 are face gears having gear tooth geometry such that the predetermined torque required to cause slipping of drive gear 188 and driven gear 190 relative to each other is the same in the clockwise and counterclockwise rotation of knob 178. Stated another way the geometry of the gear teeth 198 of drive gear 188 and gear teeth 204 of driven gear 190 results in drive gear 188 and driven gear 190 slipping relative to each upon the same application of torque by a user to knob 178 when the torquer is in the fully closed position and in the fully open position.

Upon rotation of knob 178 in the first direction shaft 164 will continue to move first pad 120 and second pad 122 toward one another to pinch the EMD until the torque required to continue to move first pad 120 toward second pad 122 exceeds a predetermined force. Once the predetermined force is reached the spring force of biasing member 196 will no longer be sufficient to maintain gear teeth 198 to impart movement to gear teeth 204. The predetermined force rotation of knob 178 in the first direction will result in gear teeth 198 sliding over gear teeth 204, providing an audible clicking sound as well as tactile haptic feedback to the user, as the first face 200 of gear teeth 198 slide up and over first face 200 of gear teeth 204. In this manner torque limiting actuator 104 acts as a clutch that limits the amount of force and torque that can be applied to the EMD as first pad 120 and second pad 122 pinch the EMD even if the operator continues to turn knob 178 in the first direction once the predetermined force is reached.

In one implementation the rotation in the first direction is a clockwise (CW) direction as is the convention for tightening/engaging the torquer to the EMD and the second direction is a counterclockwise (CCW) direction which is the convention for loosening/disengaging the torquer from the EMD. Stated another way rotating in the counter-clockwise direction will cause the pads to open. Tightening and loosening of the torque device occurs in two primary scenarios: in free space with the torque device held in the operator's hands, and mounted in the disposable cassette, with the cover closed.

Referring to FIG. 9 pad biasing member 124 includes a collar 210 having an aperture formed therein that receives a portion of shaft 164. Shaft 164 and pad biasing member 124 are free to move along torquer longitudinal axis 114 independently of one another. Pad biasing member 124 includes a first arm 212 and a second arm 214 that are spaced from one another and spaced from torquer longitudinal axis 114. First arm 212 and second arm 214 extend along the outside of pusher 112. First arm 212 includes a first branch 216 and a second branch 218 that engage with a portion on a first side of first pad 120 and second pad 122 respectively. Similarly, second arm 214 includes a first branch 220 and a second branch 222 that engage with a portion on a second side of first pad 120 and second pad 122. Where the first side and second sides of first pad 120 and second pad 122 are spaced from and are on opposite directions of torquer longitudinal axis 114. Branches 216, 218, 220 and 222 are preloaded to bias first pad 120 and second pad 122 away from one another in both an engaged position in which first pad 120 and second pad 122 pinch an EMD and in the disengaged position which first pad 120 and second pad 122 are not engaged with and pinching an EMD. In one implementation there is only one arm 212 and not a second arm 214. In one implementation more than two arms are used. Branch 216 and branch 218 are biased to push apart pad 120 and pad 122 away from one another. Each branch 216, 218, 220, and 222 include a respective bearing portion 224 (also referred to herein as bearing surface) that receives a respective post 226 extending from first pad 120 and second pad 122. Stated another way bearing portion 224 of branch 216 receives a post extending from a first side of first pad 120, bearing portion 224 of branch 218 receives a post extending from a first side of second pad 122, bearing portion 224 of branch 220 receives a post extending from a second side of first pad 120, and bearing portion 224 of branch 222 receives a post extending from a second side of second pad 122. Each post 226 extends from the first and second sides of first pad 120 and second pad 122 are free to pivot within the respective bearing portion 224. In one implementation bearing 224 is sized such that post 226 may only rotate about the post axis relative to bearing 226 and may not move in a direction along or perpendicular to torquer longitudinal axis 114 relative to bearing 226. Posts 226 have a longitudinal axis that is spaced from and perpendicular to torquer longitudinal axis 114. In one implementation the pair of posts 226 that extend from the first pad and are coaxial. Pair of posts 226 that extend from second pad 122 are coaxial and are spaced from and parallel to the longitudinal axis of posts 226 of first pad 120.

Referring to FIG. 7 and FIG. 7A proximal housing member 108 includes an annular stop surface 228 adjacent to the distal portion of threaded region 192 and adjacent to a cavity 230. Cavity 230 receives the distal portion of shaft 164 and the proximal portion of pusher 112.

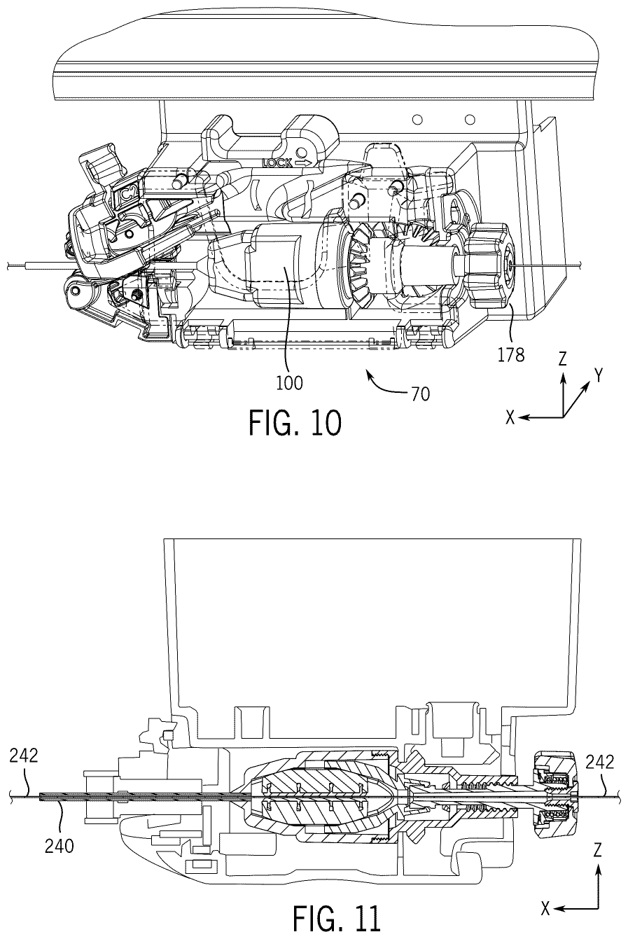

Referring to FIG. 10 and FIG. 11 torquer actuator 100 is positioned within cassette 70 such that knob 178 extends outside of cassette 70 such that knob 178 can be manipulated by a user to rotate knob 178 in both the first CW direction and the second CCW direction. Note that torquer actuator 100 includes a guide tube 240 extending from the distal end of distal housing member 110 to guide an EMD 242 through cassette 70. In one implementation knob 178 has an aperture 244 extending therethrough to permit EMD 242 to extend therethrough. Similarly, Shaft 164 also has an aperture extending therethrough to permit EMD 242 to extend therethrough.

Referring to FIG. 7 the torquer actuator 100 is moved to the fully open position by rotation of knob 178 in a counterclockwise (CCW) direction about torquer longitudinal axis 114. As knob 178 is rotated in the CCW direction the threaded portion of shaft 164 is threadedly rotated within female threaded portion of proximal housing member 108. In this manner shaft 164 is moved through proximal housing member 108 in a proximal direction. Pusher 112 being connected to distal hub 172 of shaft 164 is also moved in the proximal direction along torquer longitudinal axis 114. Shaft 164 continues to move in the proximal direction until collar 210 of pad biasing member 124 contacts annular stop surface 228 within proximal housing member 108. Once collar 210 of pad biasing member 124 contacts annular stop surface 228 shaft 164 is unable to move in the proximal direction. Further rotation of knob 178 in the CCW direction will not move shaft 164 any further in the proximal direction with respect to proximal housing member 108. When an operator continues to exert a CCW rotational force to knob 178 drive gear 188 and driven gear 190 slip with respect to one another providing an audible clicking sound and providing the user with a tactile sensation that the torquer actuator 100 is in the fully open orientation.

### NON-LIMITING ILLUSTRATIVE EMBODIMENTS

Illustrative embodiment 1. A device for manipulating an elongated medical device comprising: a torquer releasably securing an elongated medical device (EMD) thereto; and a torque limiting actuator limiting a torque applied to the torquer in a fully open position.

Illustrative embodiment 2. The device of illustrative embodiment 1, wherein the torque limiting actuator limits a torque applied to the torquer in a fully closed position.

Illustrative embodiment 3. The device of anyone of illustrative embodiment 1-2, further including a shaft that is movably positioned within a torquer housing to a fixed position relative to the torquer housing in the fully open position.

Illustrative embodiment 4. The device of anyone of illustrative embodiments 1-3, further including a member having a proximal collar that is slidably received on a shaft that is movably positioned within a torquer housing, the proximal collar prohibiting the member and a distal portion of the shaft from entering a threaded portion of the torquer housing.

Illustrative embodiment 5. The device of anyone of illustrative embodiments 1-4, further including at least a first pad having a first post and a second post extending perpendicular to a longitudinal axis of the torquer, the first pad being movable in a direction perpendicular to the first post and second post and perpendicular to the longitudinal axis of the torquer to pinch the EMD, a pad biasing member includes a first arm having a first bearing surface that receives the first post of the first pad and a second arm having a second bearing surface that receives the second post of the first pad, the first arm and the second arm biasing the first pad in a direction away from the longitudinal axis of the torquer.

Illustrative embodiment 6. The device of anyone of illustrative embodiments 1-5, further including at least a first pad movable toward and away from a longitudinal axis of the torquer to pinch and unpinch the EMD, the first pad including a first land portion spaced from and parallel to the longitudinal axis of the torquer that contacts a torquer housing that maintains the first pad in a parallel orientation to the longitudinal axis in the fully open position.

Illustrative embodiment 7. The device of anyone of illustrative embodiments 1-6, wherein the torquer includes a torquer housing having a first housing land surface, and a first pad including a first leveling surface having a first portion in contact with the first housing land surface, the first housing land surface being spaced from and substantially parallel to a longitudinal axis of the torquer.

Illustrative embodiment 8. The device of anyone of illustrative embodiments 1-7, wherein the torquer includes a pusher movable within the torquer housing to move the first pad in a direction perpendicular to the longitudinal axis of the torquer to pinch the EMD, the pusher includes at least a first pusher land surface coplanar to the first housing land surface, the first leveling surface includes a second portion in contact with the first pusher land surface when the torquer is in the fully open position.

Illustrative embodiment 9. The device of Illustrative embodiment 8, further including a second pad movable toward and away from the longitudinal axis of the torquer to pinch and unpinch the EMD between the first pad and the second pad, the second pad including a second land portion spaced from and parallel to the longitudinal axis of the torquer that contacts the torquer housing to maintain the second pad in a parallel orientation to the first pad and the longitudinal axis of the torquer in the fully open position.

Illustrative embodiment 10. The device of Illustrative embodiment 9, wherein the torquer housing includes a second housing land surface, the second pad includes a second leveling surface having a first portion in contact with the second housing land surface, the second housing land surface being spaced from and substantially parallel to the longitudinal axis of the torquer housing.

Illustrative embodiment 11. The device of Illustrative embodiment 10, wherein the pusher includes a second pusher land surface coplanar to the first housing land surface, the second leveling surface includes a second portion in contact with the second pusher land surface when the torquer is in the fully open position.

Illustrative embodiment 12. The device of illustrative embodiment 1, wherein the torque limiting actuator includes a shaft having a portion threadedly secured to a housing of the torquer and a distal portion operatively moving a pad into engagement with the EMD upon rotation of the shaft relative to the body, the shaft travels to a fixed position within a body of the torquer when the torquer is in the fully open position, a knob is releasably connected to the shaft upon an application of a predetermined torque when the torquer is in the fully open position.

Illustrative embodiment 13. The device of anyone of illustrative embodiments 1-12, wherein the knob is releasably connected to the shaft upon the application of a predetermined torque when the torquer is in a fully closed position.

Illustrative embodiment 14. The device of anyone of illustrative embodiments 1-11, wherein the torque limiting actuator limits the torque applied to the torquer to a first predetermined torque in a fully closed position and to a second predetermined torque in the fully open position.

Illustrative embodiment 15. The device of Illustrative embodiment 14, wherein the first predetermined torque is equal to or greater than the second predetermined torque, wherein the first predetermined torque and the second predetermined torque is greater than zero.

Illustrative embodiment 16. A torquer for an elongated medical device: a body having a cavity defining a pathway; a first pad movable within the cavity; a biasing member separate from the first pad biasing the first pad relative to the body; an actuator movable relative to the body moving the first pad, wherein movement of a first pad pinches and/or unpinches the elongated medical device, with the first pad, within the pathway; and a knob releasably connected to a shaft of the actuator upon an application of a predetermined torque exceeding a predetermined value in a fully open position, wherein the shaft is movable to a fixed position relative to the body in the fully open position.

Illustrative embodiment 17. The torquer of Illustrative embodiment 16, wherein the first pad including a first land portion spaced from and parallel to a longitudinal axis of the torquer that contacts a torquer housing that maintains the first pad in a parallel orientation to the longitudinal axis in the fully open position.

Illustrative embodiment 18. An EMD drive system comprising: a robotic drive having a robotic drive longitudinal axis; a device module movable along the robotic drive longitudinal axis; and a drive train coupling a motor to a driven member configured to rotate a torquer pinching an elongated medical device (EMD) about an EMD longitudinal axis, the torquer including a torque limiting actuator being manually accessible to a user when the torquer is in an in-use position in the device module; wherein the torque limiting actuator limits a torque applied to the torquer in a fully open position.

Illustrative embodiment 19. The EMD drive system of Illustrative embodiment 18, wherein the torquer includes a shaft movable within an actuator body and movable to a fixed position relative to the actuator body in the fully open position.

Illustrative embodiment 20. The EMD drive system of Illustrative embodiment 18, further including at least a first pad movable toward and away from a longitudinal axis of the torquer to pinch and unpinch the EMD, the first pad including a first land portion spaced from and parallel to the longitudinal axis of the torquer that contacts a torquer housing that maintains the first pad in a parallel orientation to the longitudinal axis in the fully open position.

Although the present disclosure has been described with reference to example embodiments, workers skilled in the art will recognize that changes may be made in form and detail without departing from the spirit and scope of the defined subject matter. For example, although different example embodiments may have been described as including one or more features providing one or more benefits, it is contemplated that the described features may be interchanged with one another or alternatively be combined with one another in the described example embodiments or in other alternative embodiments. The present disclosure described is manifestly intended to be as broad as possible. For example, unless specifically otherwise noted, the definitions reciting a single particular element also encompass a plurality of such particular elements.

What is claimed is:

1. A device for manipulating an elongated medical device comprising:
a torquer releasably securing an elongated medical device (EMD) thereto; and
a torque limiting actuator limiting a torque applied to the torquer in a fully open position.

2. The device of claim 1, wherein the torque limiting actuator limits a torque applied to the torquer in a fully closed position.

3. The device of claim 1, further including a shaft that is movably positioned within a torquer housing to a fixed position relative to the torquer housing in the fully open position.

4. The device of claim 1, further including a member having a proximal collar that is slidably received on a shaft that is movably positioned within a torquer housing, the proximal collar prohibiting the member and a distal portion of the shaft from entering a threaded portion of the torquer housing.

5. The device of claim 1, further including at least a first pad having a first post and a second post extending perpendicular to a longitudinal axis of the torquer, the first pad being movable in a direction perpendicular to the first post and second post and perpendicular to the longitudinal axis of the torquer to pinch the EMD, a pad biasing member includes a first arm having a first bearing surface that receives the first post of the first pad and a second arm having a second bearing surface that receives the second post of the first pad, the first arm and the second arm biasing the first pad in a direction away from the longitudinal axis of the torquer.

6. The device of claim 1, further including at least a first pad movable toward and away from a longitudinal axis of the torquer to pinch and unpinch the EMD, the first pad including a first land portion spaced from and parallel to the longitudinal axis of the torquer that contacts a torquer housing that maintains the first pad in a parallel orientation to the longitudinal axis in the fully open position.

7. The device of claim 1, wherein the torquer includes a torquer housing having a first housing land surface, and a first pad including a first leveling surface having a first portion in contact with the first housing land surface, the first housing land surface being spaced from and substantially parallel to a longitudinal axis of the torquer.

8. The device of claim 7, wherein the torquer includes a pusher movable within the torquer housing to move the first pad in a direction perpendicular to the longitudinal axis of the torquer to pinch the EMD, the pusher includes at least a first pusher land surface coplanar to the first housing land surface, the first leveling surface includes a second portion in contact with the first pusher land surface when the torquer is in the fully open position.

9. The device of claim 8, further including a second pad movable toward and away from the longitudinal axis of the torquer to pinch and unpinch the EMD between the first pad and the second pad, the second pad including a second land portion spaced from and parallel to the longitudinal axis of the torquer that contacts the torquer housing to maintain the second pad in a parallel orientation to the first pad and the longitudinal axis of the torquer in the fully open position.

10. The device of claim 9, wherein the torquer housing includes a second housing land surface, the second pad includes a second leveling surface having a first portion in contact with the second housing land surface, the second housing land surface being spaced from and substantially parallel to the longitudinal axis of the torquer housing.

11. The device of claim 10, wherein the pusher includes a second pusher land surface coplanar to the first housing land surface, the second leveling surface includes a second portion in contact with the second pusher land surface when the torquer is in the fully open position.

12. The device of claim 1, wherein the torque limiting actuator includes a shaft having a portion threadedly secured to a housing of the torquer and a distal portion operatively moving a pad into engagement with the EMD upon rotation of the shaft relative to a torquer housing, the shaft travels to a fixed position within the torquer housing when the torquer is in the fully open position, a knob is releasably connected to the shaft upon an application of a predetermined torque when the torquer is in the fully open position.

13. The device of claim 12, wherein the knob is releasably connected to the shaft upon the application of a predetermined torque when the torquer is in a fully closed position.

14. The device of claim 1, wherein the torque limiting actuator limits the torque applied to the torquer to a first predetermined torque in a fully closed position and to a second predetermined torque in the fully open position.

15. The device of claim 14, wherein the first predetermined torque is equal to or greater than the second predetermined torque, wherein the first predetermined torque and the second predetermined torque is greater than zero.

16. A torquer for an elongated medical device:
a body having a cavity defining a pathway;
a first pad movable within the cavity;
a biasing member separate from the first pad biasing the first pad relative to the body;
an actuator movable relative to the body moving the first pad, wherein movement of a first pad pinches and/or unpinches the elongated medical device, with the first pad, within the pathway; and
a knob releasably connected to a shaft of the actuator upon an application of a predetermined torque exceeding a predetermined value in a fully open position, wherein the shaft is movable to a fixed position relative to an actuator body in the fully open position.

17. The torquer of claim 16, wherein the first pad including a first land portion spaced from and parallel to a longitudinal axis of the torquer that contacts a torquer housing that maintains the first pad in a parallel orientation to the longitudinal axis in the fully open position.

18. An EMD drive system comprising:
a robotic drive having a robotic drive longitudinal axis;
a device module movable along the robotic drive longitudinal axis; and
a drive train coupling a motor to a driven member configured to rotate a torquer pinching an elongated medical device (EMD) about an EMD longitudinal axis, the torquer including a torque limiting actuator being manually accessible to a user when the torquer is in an in-use position in the device module;
wherein the torque limiting actuator limits a torque applied to the torquer in a fully open position.

19. The EMD drive system of claim 18, wherein the torquer includes a shaft movable within an actuator body and movable to a fixed position relative to the actuator body in the fully open position.

20. The EMD drive system of claim 18, further including at least a first pad movable toward and away from a longitudinal axis of the torquer to pinch and unpinch the EMD, the first pad including a first land portion spaced from and parallel to the longitudinal axis of the torquer that contacts a torquer housing that maintains the first pad in a parallel orientation to the longitudinal axis in the fully open position.
